(19) United States
(12) Patent Application Publication
(10) Pub. No.: US 2023/0355392 A1
(43) Pub. Date: Nov. 9, 2023

(54) ANNULOPLASTY SYSTEMS AND METHODS WITH PRELOADED TETHERS

(71) Applicant: Silara Medtech Inc., Santa Rosa, CA (US)

(72) Inventors: Trevor M. GREENAN, Santa Rosa, CA (US); Travis ROWE, Santa Rosa, CA (US); Mathew A. HAGGARD, Santa Rosa, CA (US); Do D. UONG, Santa Rosa, CA (US); Leonardo R. RODRIGUEZ, Santa Rosa, CA (US); Megan LO, Santa Rosa, CA (US); Daniel GREENAN, Santa Rosa, CA (US)

(21) Appl. No.: 18/313,228

(22) Filed: May 5, 2023

Related U.S. Application Data
(60) Provisional application No. 63/364,256, filed on May 5, 2022.

(51) Int. Cl. A61F 2/24; A61B 17/04

(52) U.S. Cl. CPC A61F 2/2466; A61B 17/0469; A61B 2017/0409; A61B 2017/0464; A61F 2220/0016; A61F 2220/0075; A61F 2230/005

(57) ABSTRACT

An exemplary method of performing an annuloplasty procedure includes introducing a catheter into a left atrium of a heart, deploying first and second members from the catheter and anchoring them to an anterior side of a mitral valve, and deploying a third member from the catheter and anchoring it to a posterior side of the mitral valve. The first member has a first flexible tensile member attached and the second member has a second flexible tensile member attached. The third member slidably tracks over the first and the second flexible tensile members when it is being deployed. Tension is applied to the first and the second tensile members to draw the first member and the second member toward the third member, thereby bringing the posterior side and the anterior side of the mitral valve annulus into closer approximation. Annuloplasty systems, devices and components are also disclosed.

### CLAIM OF PRIORITY

This application claims the benefit of U.S. Provisional Application No. 63/364,256, filed May 5, 2022, which is herein incorporated by reference in its entirety for all purposes.

### INCORPORATION BY REFERENCE

All publications and patent applications mentioned in this specification are herein incorporated by reference for all intents and purposes to the same extent as if each individual publication or patent application was specifically and individually indicated to be incorporated by reference.

### FIELD

Embodiments of the disclosure relate generally to implanted medical devices. Specifically, some implementations of the present invention relate to apparatus and methods for repairing a mitral valve.

### BACKGROUND

The mitral valve is located at the junction between the left atrium and the left ventricle of the heart. During diastole, the valve opens, in order to allow the flow of blood from the left atrium to the left ventricle. During systole, when the left ventricle pumps blood into the body via the aorta, the valve closes to prevent the backflow of blood into the left atrium. The mitral valve is composed of two leaflets (the posterior leaflet and the anterior leaflet), which are located at the mitral annulus, the annulus being a ring that forms the junction between the left atrium and the left ventricle. The mitral valve leaflets are tethered to papillary muscles of the left ventricle via chordae tendineae. The chordae tendineae prevent the mitral valve leaflets from averting into the left atrium during systole.

Mitral valve regurgitation is a condition in which the mitral valve does not close completely, resulting in the backflow of blood from the left ventricle to the left atrium. In some cases, regurgitation is caused by dilation of the mitral annulus, and, in particular, by an increase in the anteroposterior diameter of the mitral annulus. Alternatively or additionally, mitral regurgitation is causes by dilation of the left ventricle that, for example, may result from an infarction. The dilation of the left ventricle results in the papillary muscles consistently tethering the mitral valve leaflets into an open configuration, via the chordae tendineae.

Prior art methods and devices exist for treating mitral regurgitation. They involve either replacing or repairing the mitral valve. Replacing the valve is typically done either transapically or transseptally. Repairing the valve typically falls into one of four categories: leaflet clip; direct annuloplasty; indirect annuloplasty or chordae repair. Direct and indirect annuloplasty both involve reshaping the mitral annulus and or the left ventricle of a subject so that the anterior and posterior leaflet coapt properly. For some annuloplasty applications, a ring is implanted in the vicinity of (e.g., on or posterior to) the mitral annulus. The purpose of the ring is to reduce the circumference of the mitral annulus.

In light of the above prior art, it is desirable to provide improved systems and methods for treating mitral valve regurgitation.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 is a generally cranial to caudal view showing aspects of a human mitral valve;

FIG. 2 is a perspective view showing an exemplary posterior bar constructed according to aspects of the present disclosure;

FIG. 3 is a top plan view showing an exemplary anterior pad constructed according to aspects of the present disclosure;

FIG. 4 is a flowchart schematically illustrating an exemplary method of performing an annuloplasty procedure according to aspects of the present disclosure;

FIGS. 5-22 are a series of perspective views looking in a generally caudal direction through a left atrium at a mitral valve and showing the steps of the exemplary method outlined in FIG. 4;

FIG. 23 is a perspective view showing a second exemplary embodiment of an annuloplasty system constructed and implanted according to aspects of the present disclosure;

FIG. 24 is a perspective view showing the annuloplasty system of FIG. 23 with some instrumentation that may be used to implant it;

FIG. 25 is a top plan view showing the plate of the posterior implant of the annuloplasty system of FIG. 23;

FIG. 26 is a perspective view showing the posterior implant of the annuloplasty system of FIG. 23;

FIG. 27 is a top plan view showing the posterior implant of FIG. 26;

FIG. 28 is a side view showing the posterior implant of FIG. 26;

FIG. 29 is a bottom view showing the posterior implant of FIG. 26;

FIG. 30 is a perspective view of an eyelet assembly of the annuloplasty system of FIG. 23;

FIG. 31 is a side view of the eyelet assembly of FIG. 30;

FIG. 32 is a top view of the eyelet assembly of FIG. 30;

FIG. 33 is a side view of a spinner assembly of the annuloplasty system of FIG. 23;

FIG. 34 is an orthogonal side view of the spinner assembly of FIG. 33;

FIG. 35 is a bottom view of the spinner assembly of FIG. 33;

FIG. 36 is a top view of the spinner assembly of FIG. 33;

FIG. 37 is a perspective view of the posterior implant of the annuloplasty system of FIG. 23 with some instrumentation that may be used to implant it;

FIG. 38 is a perspective view showing a portion of the posterior implant and instrumentation of FIG. 37 before the anchor is implanted;

FIG. 39 is a perspective view showing a portion of the posterior implant and instrumentation of FIG. 37 after the anchor is implanted and decoupled;

FIG. 40 is a perspective view showing a portion of the posterior implant and instrumentation of FIG. 37;

FIG. 41 is an enlarged perspective view showing a portion of the posterior implant and instrumentation of FIG. 40 with some of the components shown in cross-section;

FIG. 42 is a top plan view showing the plate of an anterior implant of the annuloplasty system of FIG. 23;

FIG. 43 is a perspective view showing an anterior implant of the annuloplasty system of FIG. 23 with some instrumentation that may be used to implant it;

FIG. 44 is a side view showing the posterior implant of the annuloplasty system of FIG. 23 preloaded into an implant loader;

FIG. 45 is a side view showing an anterior implant of the annuloplasty system of FIG. 23 preloaded into an implant loader;

FIG. 46 is a side view showing the anterior implant being deployed from the implant loader of FIG. 45;

FIG. 47A is a flowchart schematically illustrating a second exemplary method of performing an annuloplasty procedure according to aspects of the present disclosure; and

FIG. 47B is a perspective view showing the annuloplasty system of FIG. 23 implanted across a mitral valve according to an exemplary procedure of the present disclosure.

### DETAILED DESCRIPTION

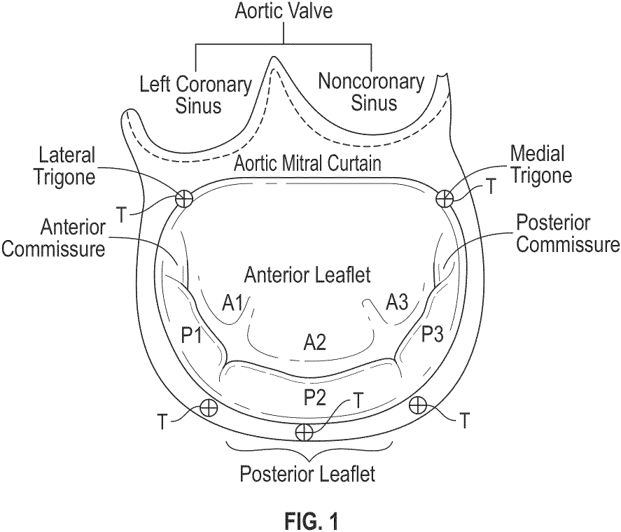

Referring to FIG. 1, elements of a mitral valve are shown. In particular, the mitral valve comprises an anterior leaflet, a posterior leaflet, an anterior-lateral commissure, a posterior-medial commissure, a lateral trigone (sometimes referred to as left) and a medial trigone (sometimes referred to as right). The anterior leaflet includes three divisions A1, A2 and A3. Similarly, the posterior leaflet also includes three divisions P1, P2 and P3. According to aspects of the present disclosure, in some implementations device anchors may be placed at or near each of the target locations T as shown.

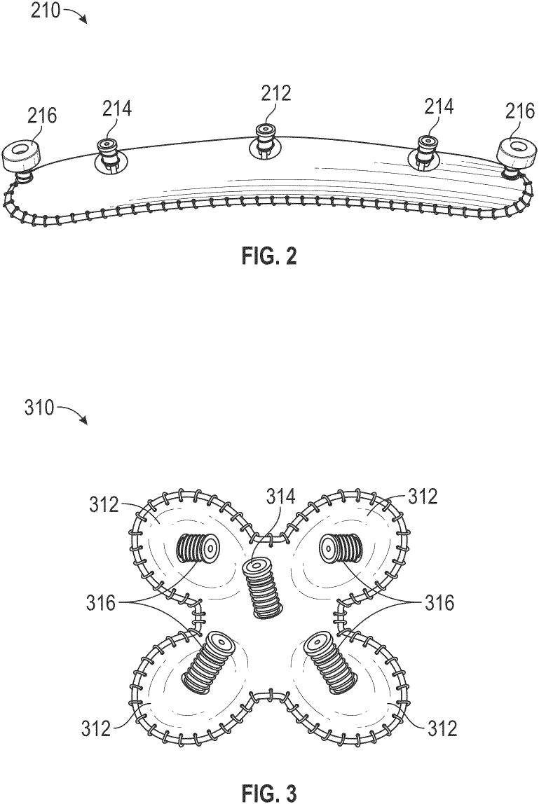

Referring to FIG. 2, an exemplary posterior bar 210 constructed according to aspects of the present disclosure is shown. Posterior bar 210 is configured to be implanted in the left atrium on or near the mitral annulus adjacent to the posterior leaflet, as will be subsequently described in more detail. As such, in this exemplary embodiment, posterior bar 210 is an elongated tubular structure that is curved to match the anatomy of the mitral annulus in this location. Posterior bar 210 may be provided with a low profile as shown to minimize the amount of irregular structure in the atrium that might be a potential site for thrombosis. In this exemplary embodiment, posterior bar 210 is provided with atraumatic edges to limit the potential for tissue damage, and is covered in polyethylene terephthalate (PET) fabric to aid with tissue ingrowth.

In this exemplary embodiment, posterior bar 210 is provided with a middle tissue anchor guide 212 and two end tissue anchor guides 214. In some embodiments, middle tissue anchor guide 212 is identical to end tissue anchor guides 214, and in other embodiments it is configured differently, such as having features that facilitate the steering/torquing of posterior bar 210 during delivery. In some embodiments, as will be subsequently described herein, there may be no middle tissue anchor guide, and there may be greater or fewer than the three tissue anchor guides provided in this exemplary embodiment. Anchor guides 212 and 214 may be configured to pivot relative to posterior bar 210 such that they can move from a retracted state and a deployed state. In the retracted state, anchor guides 212 and 214 may extend generally parallel to bar 210 so that they and bar 210 may together pass through a lumen of a catheter. In the deployed state, anchor guides 212 and 214 may extend generally perpendicular to bar 210 as shown in FIG. 2 so that they may be used to thread a tissue anchor over the guide, through apertures in bar 210 and into adjoining tissue to secure bar 210 to the tissue.

One or more snare features 216 may be provided on posterior bar 210. In this exemplary embodiment, two snare features 216 are provided, one near each end of posterior bar 210. Snare feature 216 may be configured to prominently extend from posterior bar 210 such that they can easily engage with one or more tensile members/snares, and also to prevent the tensile members from disengaging during manipulation. In some embodiments, snare features 216 are configured to be easily imaged under fluoroscopy and echocardiography to aid in positioning posterior bar 210 during delivery and attachment to tissue, and to aid in connecting tensile members to the snare features 216.

Posterior bar 210 may be designed to preferentially load anchors in shear versus tension with respect to the anatomy. Torque control features may be provided to allow the initial positioning of posterior bar 210, and to allow the ability to move the implant as subsequent anchors are delivered to match the anatomy.

Posterior bar 210 may also be provided with some level of flexibility to allow for in vivo adjustment of the bar to contour to the particular subject’s anatomy. The flexibility of posterior bar 210 may also serve to allow the bar to flex during the cardiac cycle. In some embodiments, the flexibility of posterior bar 210 is created by providing a series of slits (not shown in FIG. 1) transverse to the longitudinal axis of the bar. In some embodiments, the slits and or other flexibility-providing features may be configured to limit the minimum radius of posterior bar 210 when implanted to ensure it applies a more uniform tension to the posterior side of the mitral annulus.

Referring to FIG. 3, an exemplary anterior pad 310 constructed according to aspects of the present disclosure is shown. Anterior pad 310 is configured to be implanted in the left atrium on or near the mitral annulus adjacent to the anterior leaflet, particularly on a trigone, as will be subsequently described in more detail. In this exemplary embodiment, anterior pad 310 is a generally flat structure provided with four petals 312 radially extending from a central portion. In other embodiments, more, fewer or no petals may be provided. A primary tissue anchor 314 may be located in the center of anterior pad 310. In some embodiments, additional tissue anchors 316 may be provided, such as an additional anchor 316 near the center of each petal 312, as shown. In some embodiments, primary tissue anchor 314 is identical to additional tissue anchors 316, and in other embodiments it is configured differently, such as having features that facilitate the positioning of anterior pad 310 during delivery. The petals 312 may be designed to fold into a compact configuration such that anterior pad 310 may be delivered through a catheter.

Anterior pad 310 may be provided with a low profile as shown to minimize the amount of irregular structure in the atrium that might be a potential site for thrombosis. In this exemplary embodiment, anterior pad 310 is provided with atraumatic edges to limit the potential for tissue damage, and is covered in polyethylene terephthalate (PET) fabric to aid with tissue ingrowth.

One or more snare features may be provided on anterior pad 310. In this exemplary embodiment, the top ends of tissue anchors 314 and 316 are configured to engage with one or more tensile members/snares These snare features may be configured to prominently extend from anterior pad 310 such that they can easily engage with one or more tensile members/snares, and also to prevent the tensile members from disengaging during manipulation. In some embodiments, the snare features and or the entire anterior pad 310 are configured to be easily imaged under fluoroscopy and echocardiography to aid in positioning anterior pad 310 during delivery and attachment to tissue, and to aid in connecting tensile members to the snare features. Anterior pad 310 may be designed to preferentially load anchors in shear versus tension with respect to the anatomy.

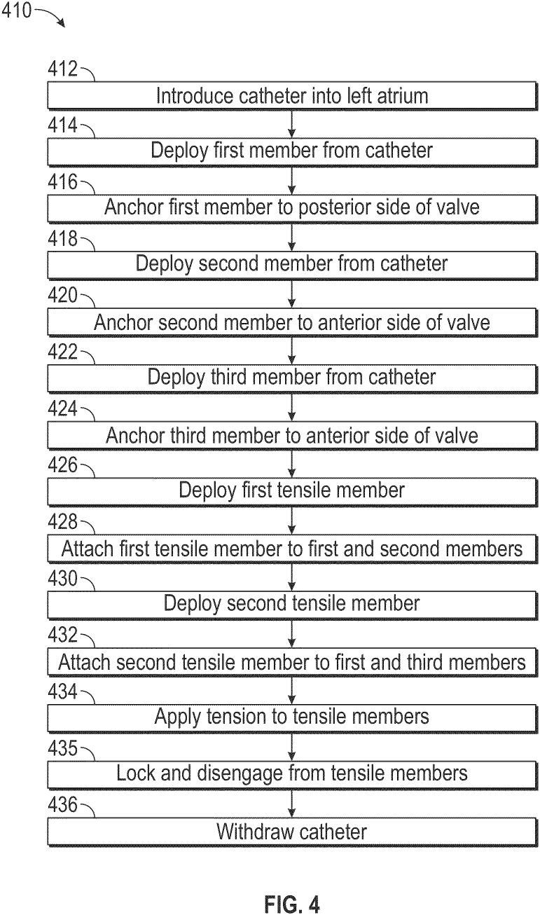

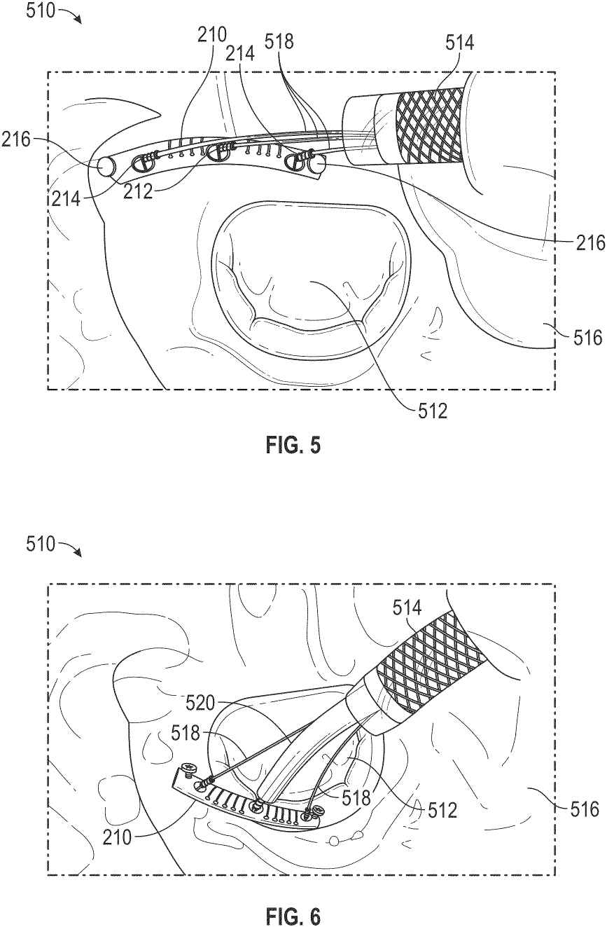

Referring to FIG. 4, an exemplary method of performing an annuloplasty procedure according to aspects of the present disclosure is shown. The steps of this exemplary method 410 will be described in reference to the flowchart shown in FIG. 4 and the series of images shown in FIGS. 5-22. In each of the images shown in FIGS. 5-22, the view is looking in a generally caudal direction through the left atrium 510 toward the mitral valve 512 with the medial direction generally to the right. In some implementations of the method, one posterior bar 210 and one, two or more anterior pads 310 are implanted. In other implementations, different types or numbers of devices may be used. In FIGS. 5-22, posterior bar 210 is shown without a fabric cover for clarity. In this exemplary embodiment, at least one device anchor is placed at or near each of the five target locations T shown in FIG. 1.

In some implementations of method 410, the first step 412 of the method is introducing the distal end of a delivery catheter into the left atrium 510 of a subject. This may be performed using a transseptal approach, a left atrial approach or other methodology for gaining access to the left atrium. In the images shown in FIGS. 5-22, a transseptal approach is depicted with the distal end of catheter 514 passing through the septum 516 of the heart and into the left atrium 510 of the subject. In some implementations, an inner dilator (not shown) is located in the distal end of catheter 514 for crossing the septum.

Referring to FIGS. 4 and 5, once the distal end of catheter 514 is introduced into the left atrium 510, a posterior bar 210, sometimes referred to herein as a first member, may be deployed from the distal end of catheter 514 in step 414. In some implementations, catheter 514 is first introduced into the left atrium 510 before the posterior bar assembly is loaded into the proximal end of the catheter 514. In other implementations the posterior bar 210 along with its tissue anchor guides 212, 214 and snare features 216 may be pre-loaded into catheter (not shown) and advanced through catheter 514. As seen in FIG. 5, an anchor lead 518 may be removably attached to each of the tissue anchor guides 212 and 214 to push posterior bar 210 through catheter 514 and deploy it from the distal end.

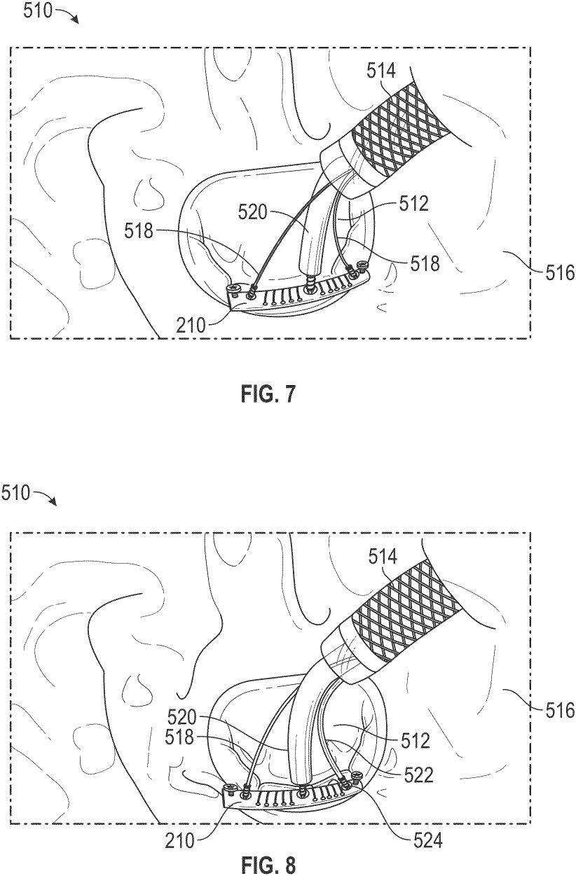

Referring to FIG. 6, once posterior bar 210 emerges from the distal end of catheter 514, a lead 518 attached to one of its ends may be pushed and the other pulled from the proximal end of catheter 514 to pivot posterior bar 210 into an orientation that is generally perpendicular to catheter 514, as shown. A steerable inner catheter 520 may be slid distally over middle lead 518 until features (such as recesses and/or castellations, not shown) engage with mating features on posterior bar 210 to keep bar 210 from rotating relative to inner catheter 520. Steerable inner catheter 520 may then be used to position and rotate posterior bar 210 until it is steered into its desired implantation location and orientation, as shown in FIG. 7. In some implementations, a torque driver coaxially located between lead 518 and steerable inner catheter 520 may be used to impart torque to posterior bar 210. Such an implementation is subsequently described in relation to FIGS. 40-46.

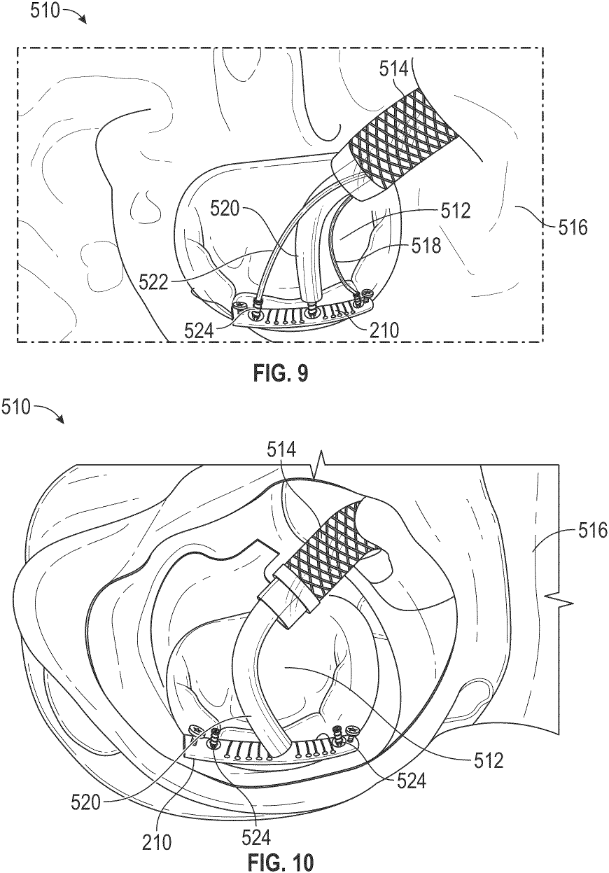

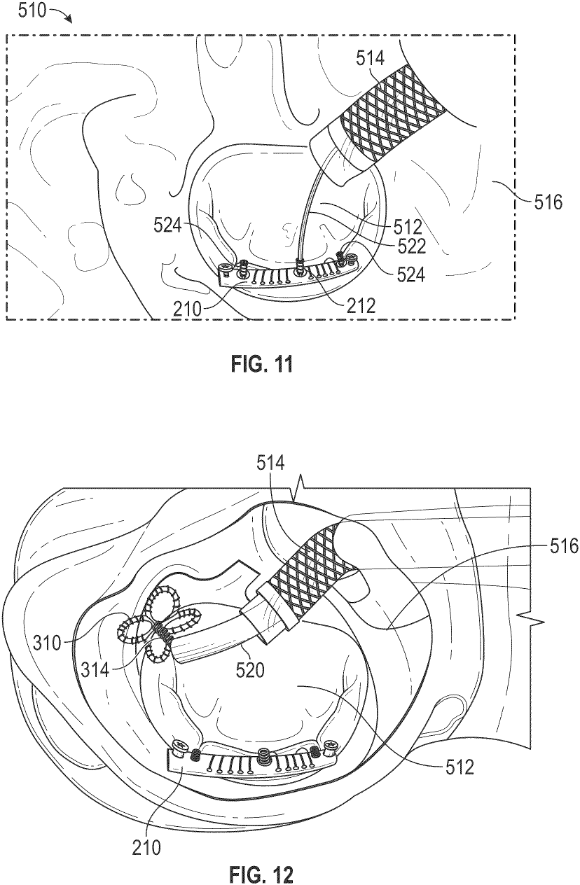

Referring to FIGS. 4 and 8-11, step 416 of exemplary method 410 will be described. In this step, posterior bar 210 (i.e. the first member) is anchored to the posterior side of mitral valve 512. This may be accomplished by first sliding a drive tube 522 with a helical tissue anchor 524 located on its distal end over lead 518 attached to the tissue anchor guide 214 located near the medial end of posterior bar 210, as shown in FIG. 8. While steerable inner catheter 520 holds posterior bar 210 against the mitral valve annulus tissue, drive tube 522 may be rotated to screw medial anchor 522 through posterior bar 210 and into the underlying tissue, as seen in FIG. 9. Drive tube 522 may then be removed from the medial anchor 214 and it (or another drive tube 522 with another helical tissue anchor 524) may be slid over lead 518 attached to the tissue anchor guide 214 located near the lateral end of posterior bar 210, as shown in FIG. 9. While medial anchor 524 and steerable inner catheter 520 (and in some implementations a torque driver inside catheter 520) hold posterior bar 210 against the mitral valve annulus tissue, drive tube 522 may be rotated to screw lateral tissue anchor 524 through bar 210 and into the underlying tissue, as seen in FIG. 10. Drive tube 522 may then be removed from the lateral anchor 524 and it (or another drive tube 522 with another helical tissue anchor 524) may be slid over lead 518 attached to the middle tissue anchor guide 212, as shown in FIG. 11. In some implementations, steerable inner catheter 520 may remain in place against posterior bar 210 when the center anchor is being placed (as shown in FIG. 10), or it may be removed from posterior bar 210 prior to drive tube 522 and middle anchor 524 being slid into engagement over middle tissue anchor guide 212 (as shown in FIG. 11.) While medial and lateral anchors 524 hold posterior bar 210 against the mitral valve annulus tissue, drive tube 522 may be rotated to screw middle anchor 524 through bar 210 and into the underlying tissue. FIGS. 10 and 11 show posterior bar 210 with the leads removed from the end tissue anchor guides, such as by unthreading.

In step 416, it should be noted that after the initial anchor has been placed, torque control of the implant 210 provided by steerable inner catheter 520 (or in some implementations a torque driver located within catheter 520) can be used to guide the placement of subsequent anchors to implant 210. This eliminates the need for unguided anchor placement after the initial anchor has been placed. FIG. 12 shows posterior bar 210 with the three anchors placed and all leads removed.

Referring to FIGS. 4 and 12, step 418 of exemplary method 410 will be described. In this step, anterior pad 310 (sometimes referred to herein as a second member) is deployed from the distal end of catheter 514. In some implementations, anterior pad 310 is steered toward the lateral trigone with steerable inner catheter 520 as shown in FIG. 12. (The lateral trigone is also shown in FIG. 1.)

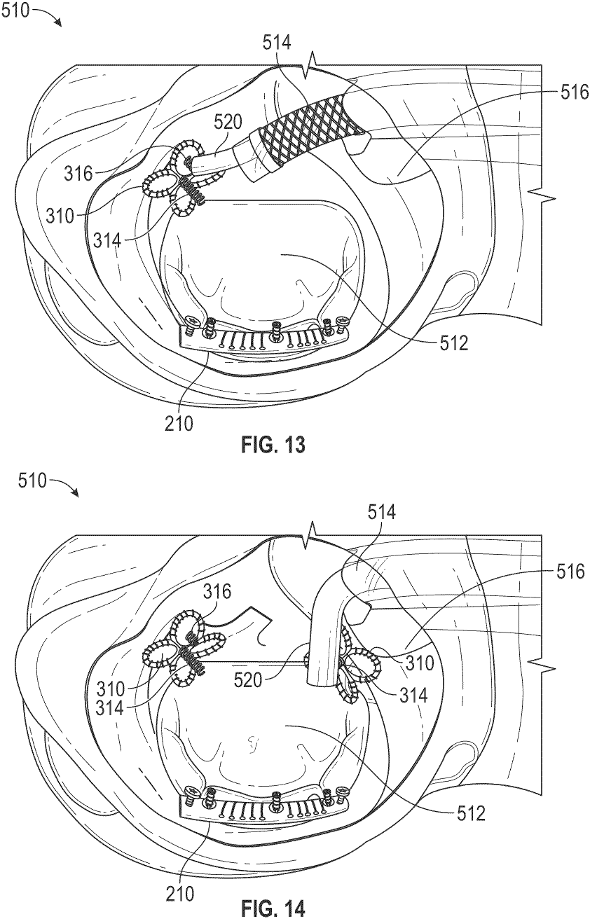

Referring to FIGS. 4, 12 and 13, step 420 of exemplary method 410 will be described. In this step, anterior pad 310 (sometimes referred to herein as a second member) is anchored to the anterior side of mitral valve 512. In some implementations, anterior pad 310 is anchored to the lateral trigone as shown with a single anchor 314. A drive tube (not shown) may be used within steerable inner catheter 520 to screw anchor 314 into place. As shown in FIG. 13, additional anchor(s) 316 may be used to further secure anterior pad 310 to the lateral trigone.

In step 420, it should be noted that after the initial anchor has been placed, its lead can remain in place through steerable inner catheter 520 such that the lead and catheter 520 can be used to guide the placement of subsequent anchors to implant 310. This eliminates the need for unguided anchor placement after the initial anchor has been placed.

Referring to FIGS. 4 and 14, steps 422 and 424 of exemplary method 410 will be described. In these steps, another anterior pad 310 (sometimes referred to herein as a third member) is deployed from the distal end of catheter 514. In some implementations, anterior pad 310 is steered toward the medial trigone with steerable inner catheter 520 as shown in FIG. 14. (The medial trigone is also shown in FIG. 1.) Anterior pad 310 may then anchored to the anterior side of mitral valve 512. In some implementations, anterior pad 310 is anchored to the medial trigone as shown with a single anchor 314. A drive tube (not shown) may be used within steerable inner catheter 520 to screw anchor 314 into place. As with the lateral anterior pad 310, additional anchor(s) may be used to further secure the medial anterior pad 310 to the medial trigone.

In step 424, it should be noted that after the initial anchor has been placed, its lead can remain in place through steerable inner catheter 520 such that the lead and catheter 520 can be used to guide the placement of subsequent anchors to implant 310. This eliminates the need for unguided anchor placement after the initial anchor has been placed.

Referring to FIGS. 4 and 15, step 426 of exemplary method 410 will be described. In this step, a first tensile member, tether or snare 526 is deployed from the distal end of catheter 514 through steerable inner catheter 520 as shown. A snare sheath 528 may be used to direct the first tensile member 526 toward implant features. Snare sheath 528 may also be used to tighten first tensile member 526 around the implant features by pulling proximally on the tensile member 526 relative to the sheath 528.

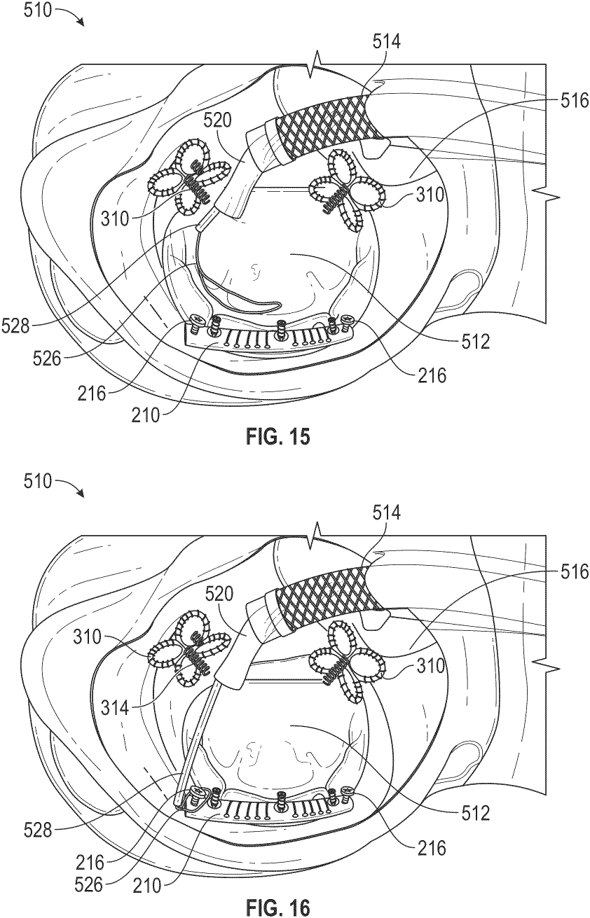

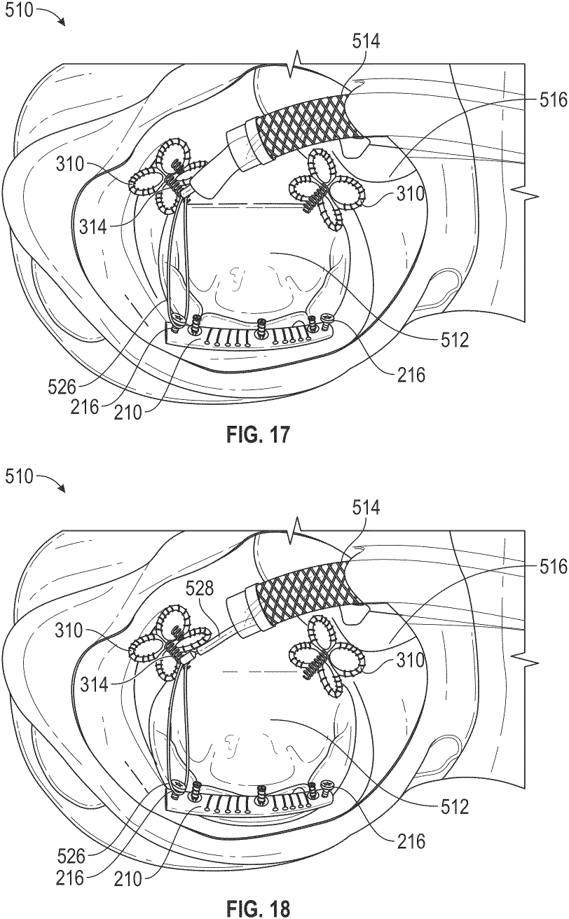

Referring to FIGS. 4 and 16-18, step 428 of exemplary method 410 will be described. In this step, first tensile member or snare 526 is attached to the posterior bar 210 (i.e. the first member) and anterior pad 310 (i.e. the second member.) Steerable inner catheter 520 and snare sheath 528 may be utilized to guide first tensile member 526 over the lateral snare feature 216 of bar 210, as shown in FIG. 16. First tensile member 526 may then be guided over primary tissue anchor 314 of anterior pad 310, as shown in FIG. 17. A small amount of tension may then be applied to first tensile member 526 with snare sheath 528 to keep it engaged with bar 210 and pad 310, as shown in FIG. 18.

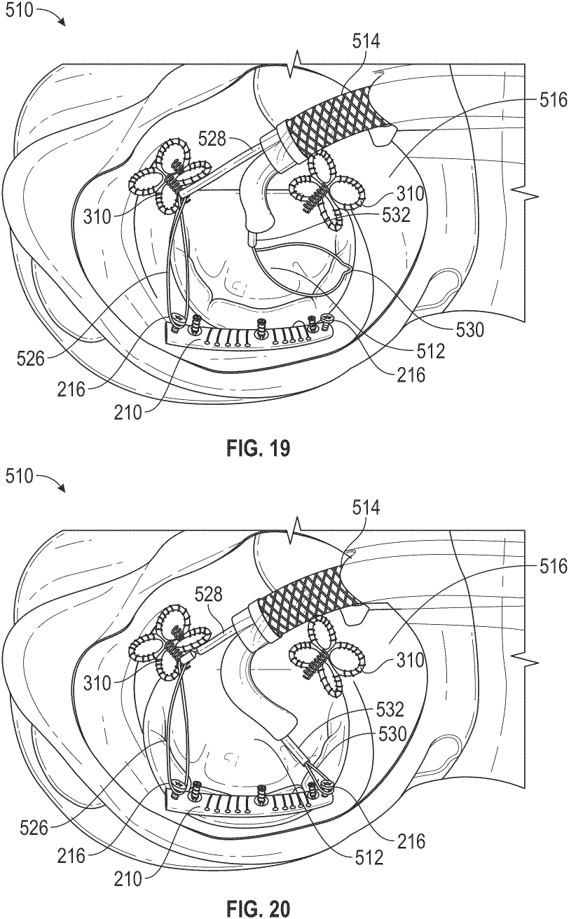

Referring to FIGS. 4 and 19, step 430 of exemplary method 410 will be described. In this step, a second tensile member or snare 530 is deployed from the distal end of catheter 514 through steerable inner catheter 520 as shown. A snare sheath 532 may be used to direct the second tensile member 530 toward implant features. Snare sheath 532 may also be used to tighten second tensile member 530 around the implant features by pulling proximally on the tensile member 530 relative to the sheath 532.

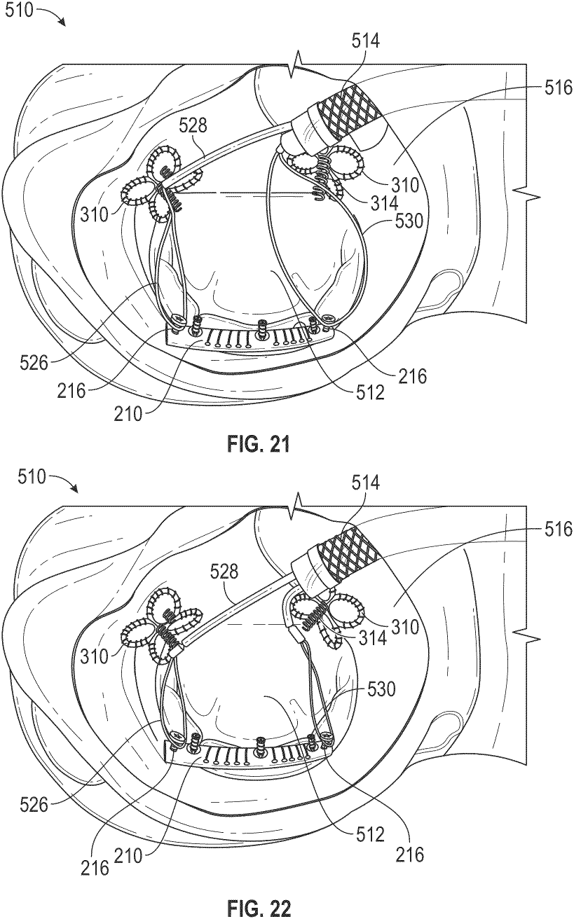

Referring to FIGS. 4 and 20-22, step 432 of exemplary method 410 will be described. In this step, second tensile member or snare 530 is attached to the posterior bar 210 (i.e. the first member) and the next anterior pad 310 (i.e. the third member.) Steerable inner catheter 520 and snare sheath 532 may be utilized to guide second tensile member 530 over the medial snare feature 216 of bar 210, as shown in FIG. 20. Second tensile member 530 may then be guided over primary tissue anchor 314 of anterior pad 310, as shown in FIG. 21. A small amount of tension may then be applied to second tensile member 530 with snare sheath 532 to keep it engaged with bar 210 and pad 310, as shown in FIG. 22. In some implementations, the snare shape may be configured to more easily engage the snare features on the implants. For example, each snare may form a D-shape that makes contact with the lateral side or medial side of the atrium. The wall of the atrium is then used to guide the snare down to the annulus and then cinch without necessarily needing to guide the snare to each snare feature. In some embodiments, the snare has a dumbbell (or dog bone) shape, such as the exemplary snare 550 shown in FIG. 54. Snare 550 includes a distal loop 552 and a proximal loop 554 having predefined diameter(s), with the rest of the snare having generally parallel tensile members forming a smaller gap between them than the loop diameter(s). Distal loop 552 may first be exposed to engage a first snare feature on an implant, and subsequently the proximal loop 554 may be exposed to capture a second snare feature on an implant. In some embodiments, as depicted in FIG. 55, two snares 560 and 562 are loaded in parallel, each with a predefined shape. Individual snares 560 and 562 may be connected with a coupler 564 and can slide independently to engage snare features on implants separately.

Once both first tensile member 526 and second tensile member 530 are in place, additional tension may be applied to both to draw the anterior and posterior sides of mitral valve 512 into closer approximation. In some implementations, tension in members 526 and 530 may be increased simultaneously. In some implementations, tension may be increased incrementally in members 526 and 530, alternating between the two until the desired tensions and or valve approximation is reached. In some implementations, the final tension and or tissue approximation of each tensile member 526 and 530 is approximately the same. In some implementations, the final tension and or tissue approximation of each tensile member 526 and 530 is different. Because medial and lateral cinching can be performed independently, the placement of each bar is more forgiving. This generally holds true for all of the systems disclosed herein. In some implementations, real time echocardiography of the mitral valve is used to monitor the reduction in mitral regurgitation as tensile members 526 and 530 are tightened.

After the desired tensions and or tissue approximations are obtained, tensile members 526 and 530 may be tied off. In some implementations, a reversible lock may be used during the cinching process which is configured to permanently hold the position of the tensile member. A disconnect member may be used to decouple the snare from the delivery system, or a portion of the tensile member may be cut to release it. Catheter 514 may then be withdrawn from the left atrium, along with steerable inner catheter 520 and snare sheaths 528 and 532 (step 436 shown in FIG. 4.) In addition to tensioning the device during the de novo procedure, additional tensioning devices can be added at a later time or date, and or the existing devices can be re-tensioned to further reduce the A-P dimension.

Additional embodiments of the preceding system and method can be found in Applicant’s co-pending U.S. Pat. Application Publication 2021/0052387, entitled Annuloplasty Systems and Methods.

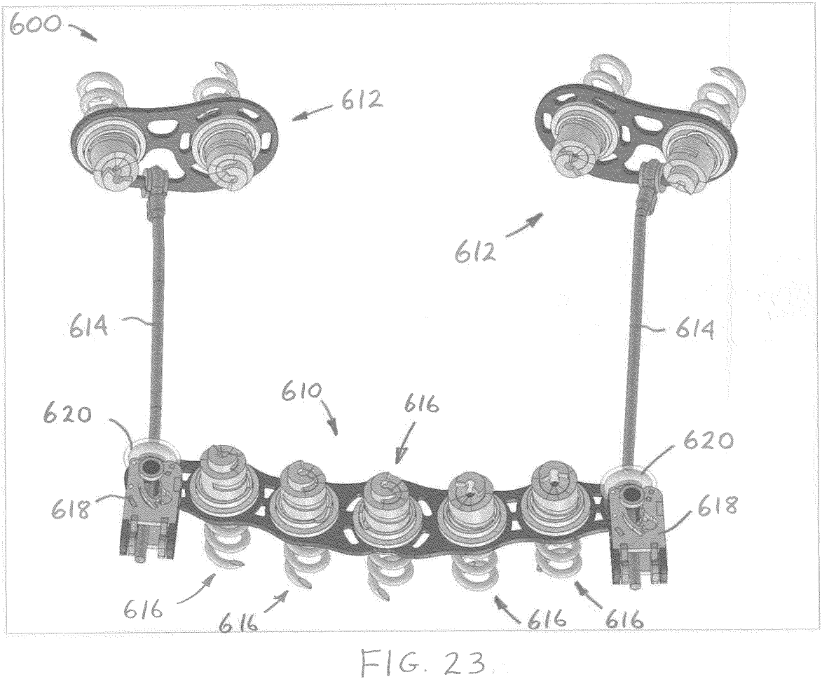

Referring to FIG. 23-47B, a second exemplary embodiment of an annuloplasty system 600 constructed and implanted according to aspects of the present disclosure is shown. Referring first to FIG. 23, annuloplasty system 600 is constructed and functions in a manner similar to the previously described system. It too includes an elongated posterior implant 610 configured to be implanted in the left atrium on or near the mitral annulus adjacent to the posterior leaflet, and two anterior implants 612 each configured to be implanted in the left atrium on or near the mitral annulus adjacent to the anterior leaflet, particularly on a trigone. However, in this second exemplary embodiment, instead of attaching tensile members or tethers to the implants after they have been deployed, tethers 614 are pre-attached to anterior implants 612 before they are deployed from a catheter. The anterior implants 612 are implanted first in this embodiment, and their tethers 614 are threaded through the posterior implant 610 before it is deployed from its catheter. The posterior implant 610 is then deployed from its catheter and tracks over tethers 614 as it is placed on the posterior side of the mitral annulus. After the posterior implant 610 is secured in place with anchors 616, tethers 614 can be tensioned and secured with locks 618, as will be subsequently described in detail. This arrangement saves considerable time during the procedure, not needing to snare each of the implants with the tethers. It also ensures the tether attachment points are more consistent and reliable.

In this second exemplary embodiment, posterior implant 610 is provided with five anchors 616 and anterior implants 612 are each provided with two anchors 616. Each end of posterior implant 610 is provided with a swiveling eyelet assembly 620 for tracking over a tether 614 and providing a backstop for a tether lock 618.

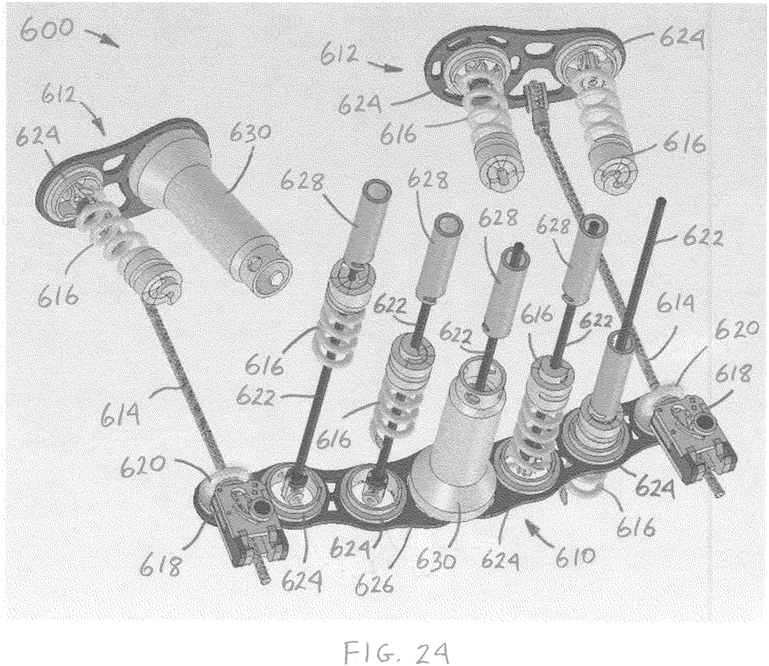

Referring to FIG. 24, another perspective view of system 600 is provided, showing anchors 616 in various stages of insertion. Each anchor 616 is guided into place by its own lead 622 detachably connected to a spinner assembly 624. Each spinner assembly 624 is rotatably attached to posterior implant plate 626. A separate driver head 628 is detachably connected to the top of each anchor 616. When a driver head 628 is rotated by a proximally extending driver tube (not shown), the attached anchor 616 is driven through its spinner assembly 624 and into the underlying heart tissue until it seats against its spinner assembly 624, thereby securing implant plate 626 against the tissue. A torque head 630 is provided for each implant (only two are shown in FIG. 24.) Each torque head 630 is longitudinally and rotationally driven by a proximally extending torque tube (not shown) to engage with its respective implant and drive the implant into position for anchoring.

Referring to FIG. 25, a bare posterior implant plate 626 is shown without any components attached for clarity. It is provided with five through holes 632 for rotatably retaining spinner assemblies 624 (shown in FIGS. 23 and 24.) Two holes 634 are also provided for rotatably retaining eyelet assemblies 620 (shown in FIGS. 23 and 24.) A series of slots 636 spaced around the center spinner assembly hole 632 are provided for engaging with torque head 630 (shown in FIGS. 23 and 24), as will be subsequently described in detail. Additional through holes 638 and scallops 640 may be provided as shown to reduce the amount of metal in plate 626 for better echo imaging and tissue ingrowth.

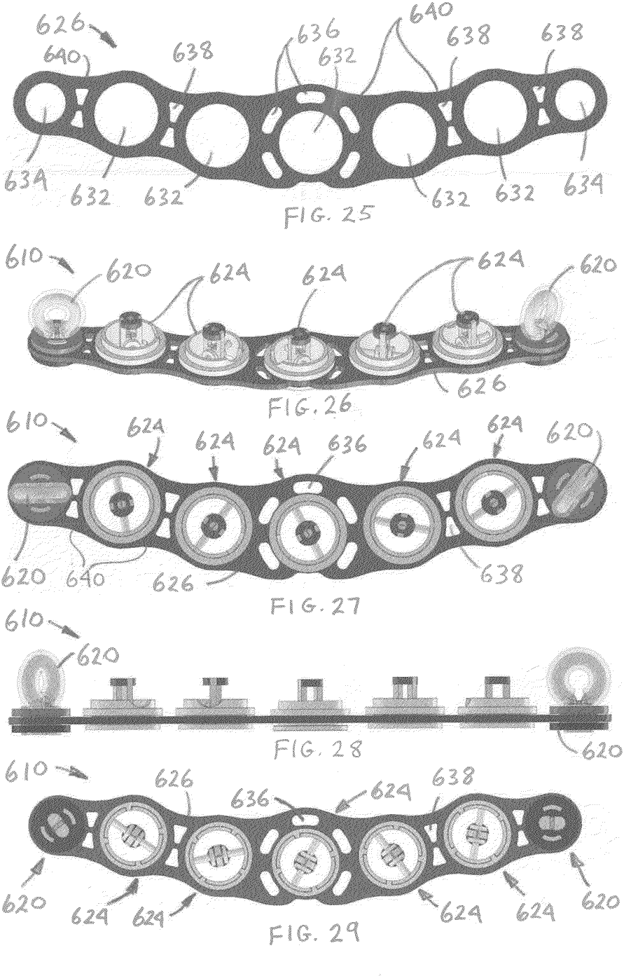

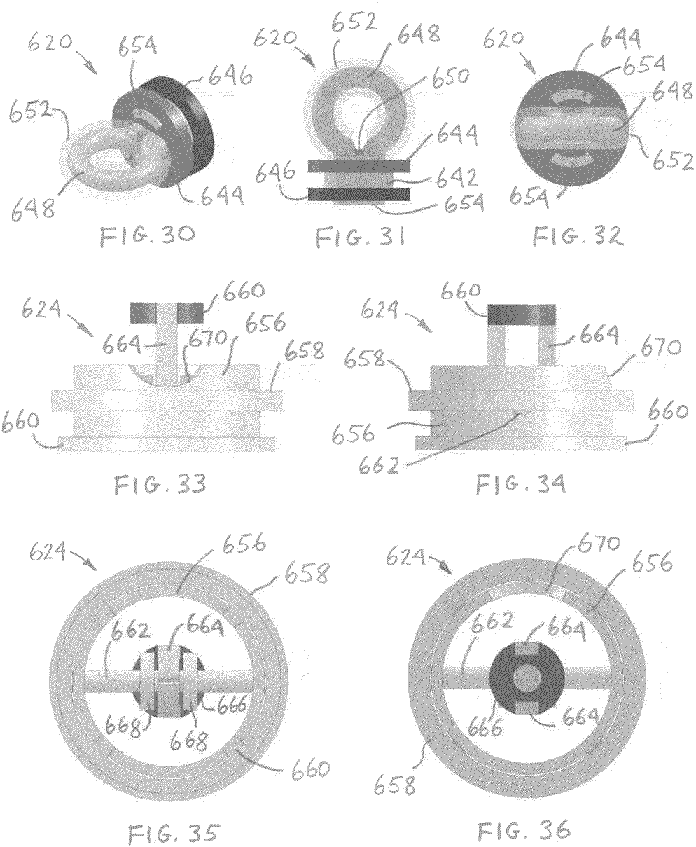

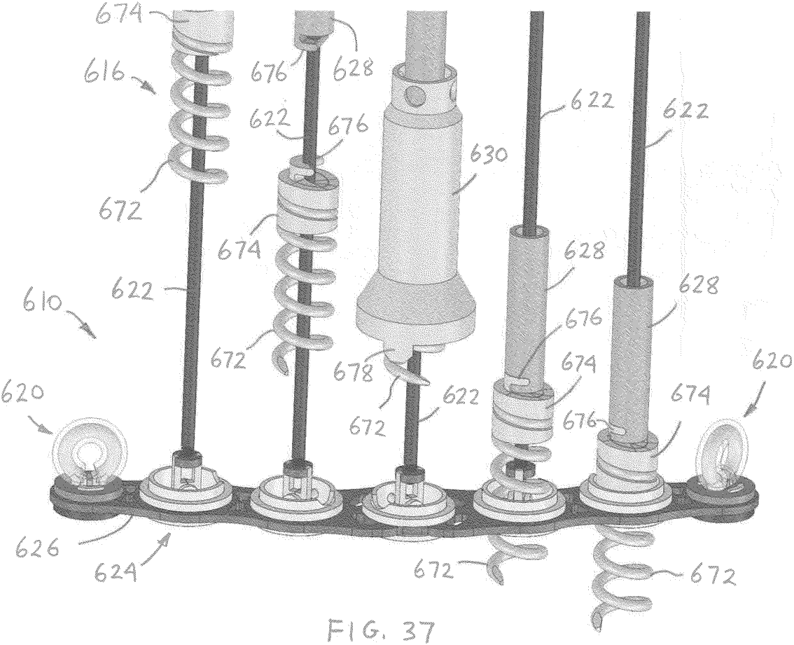

Referring to FIGS. 26-29, posterior implant plate 626 is shown with anchor spinner assemblies 624 and tether eyelet assemblies 620 attached to base plate 626. FIG. 26 is a perspective view, FIG. 27 is a top plan view, FIG. 28 is a side view and FIG. 29 is a bottom view.

Referring to FIGS. 30-32, various views of eyelet assembly 620 are shown. FIG. 30 is a perspective view, FIG. 31 is a side view and FIG. 32 is a top plan view. (A bottom view of eyelet assembly 620 is provided in FIG. 29.) As best seen in FIG. 31, eyelet assembly 620 may be formed from six separate components: a cylindrical core 642, a top ring 644, a bottom ring 646, an eyelet 648, a wedge or filler material 650 and an eyelet covering 652. As best seen in FIGS. 31 and 32, core 642 may be provided with two pairs of arcuate fins 654, one pair protruding from the top of core 642 and one pair protruding from the bottom. Top ring 644 and bottom ring 646 may each be provided with mating slots for receiving the arcuate fins 654. In some embodiments, fins 654 are swaged, welded, epoxied, press-fit and or fastened to rings 644 and 646 by other suitable means. In other embodiments, there is only a sliding fit between fins 654 and rings 644 and 646, and the rings are held in place by being sandwiched between core 642 and the ends of a straight shank portion of eyelet 648, and or by the eyelet shank expanding against the rings, as will be described next. In some embodiments, fins 654 may serve as centering features for core 642, and or as anti-rotation features such that rings 644 and 646 do not rotate relative to core 642 and or eyelet 648. In other embodiments (not shown), feature shapes other than arcuate fins may be used.

Eyelet 648 may be formed with a straight shank section that has an oval or oblong transverse cross-section (best seen in FIG. 29.) Core 642 may be provided with a central bore having a mating oval or oblong transverse cross-section or a circular cross-section for receiving the eyelet shank. The straight shank section may be split down the center, with a gap between the two halves. This allows an anti-friction tube 652 to be slid over one half of the shank and onto the circular portion of eyelet 648, as shown. In some embodiments, tube 652 is made of or coated with polytetrafluoroethylene (PTFE) to reduce friction between eyelet 648 and the tether that passes through it. In other embodiments, eyelet 648 may be dipped directly into PTFE or another friction-reducing coating.

During assembly, the straight shank section of eyelet 648 may be passed through top ring 644, the center of core 642 (which resides in one of the holes 634 of posterior implant plate 626, shown in FIG. 25) and through bottom ring 646. A wedge or filler material 650 may then be placed between the two halves of the eyelet shank such that they are urged outwardly against the inside walls of oval or oblong bores within rings 644 and 646. In some embodiments, material 650 is a resiliently compressible material placed in the shank gap before assembly, such that it can be compressed during assembly and then exert a resilient outward force after assembly. In other embodiments, material 650 is a metal (i.e. non-compressible.) In some embodiments, the eyelet shank is provided with a necked-down portion (not shown) having an axial length slightly longer than the distance between the top of top ring 644 and the bottom of bottom ring 646. This arrangement allows core 642 and rings 644 and 646 to be captivated within the necked-down portion after the split shank is radially compressed, passed through the other components and then radially expanded. Once eyelet assembly 620 is assembled, posterior implant plate 626 (shown in FIG. 25) is sandwiched between top ring 644 and bottom ring 646 of eyelet assembly 620. Core 642 may be provided with a height that is slightly larger than the thickness of implant plate 626 such that eyelet assembly 620 may freely rotate relative to plate 626.

Each of the exemplary embodiments described above provide eyelet assembly 620 with the ability to rotate relative to posterior implant 610, thereby allowing the tether passing through the eyelet assembly 620 to align with a central lumen of a catheter during delivery and then rotate to align with an anterior trigone implant 612 once implanted.

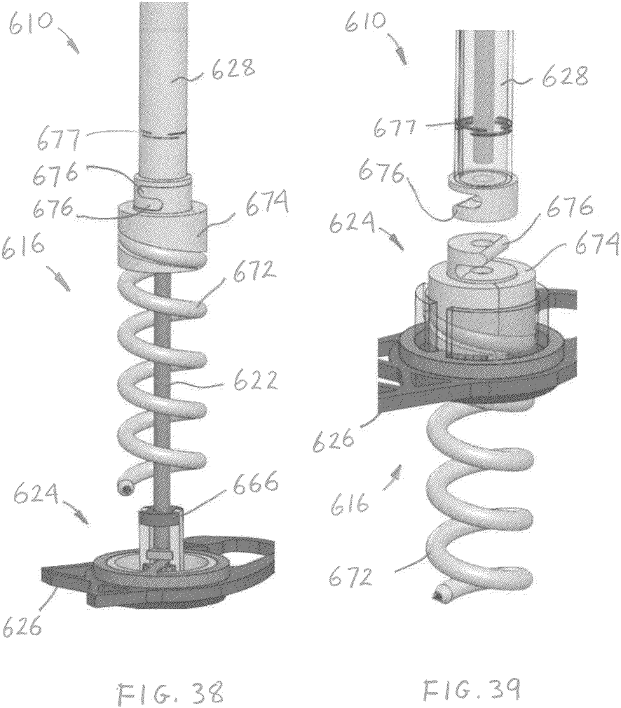

Referring to FIGS. 33-36, various views of spinner assembly 624 are shown. FIG. 33 is a side view, FIG. 34 is another side view, taken in a direction orthogonal to that of FIG. 33, FIG. 35 is a bottom view and FIG. 36 is a top view. Spinner assembly 624 may be formed from six separate components: a central hoop 656, a top disc 658, a bottom lock ring 660, a crossbar 662, a U-shaped connecting rod 664 and a lead nut 666. Central hoop 656 is configured to be rotatably received within one of holes 632 in posterior implant plate 626 (shown in FIG. 25.) Disc 658 and ring 660 may each be welded or otherwise connected to hoop 656 to rotatably captivate implant plate 626 therebetween. Crossbar 662 spans across a central bore in hoop 656 (parallel to plate 626.) U-shaped connecting rod 664 is pivotably mounted to crossbar 662, as best seen in FIG. 35 (and also shown in FIG. 41.) Lead nut 666 can be welded or otherwise fastened to the top of connecting rod 664. Lead nut 666 is provided with a central threaded bore for receiving a threaded end of an anchor lead 622 (shown in FIGS. 24 and 37.) Fixed flanges 668 may be provided on crossbar 662 to keep connecting rod 664 and lead nut 666 centered within hoop 656. Connecting rod 664 may be laser cut in an open (V-shaped) configuration and then closed (made into a U-shape) around crossbar 662 between flanges 668 before nut 666 is attached to the tops of its two prongs.

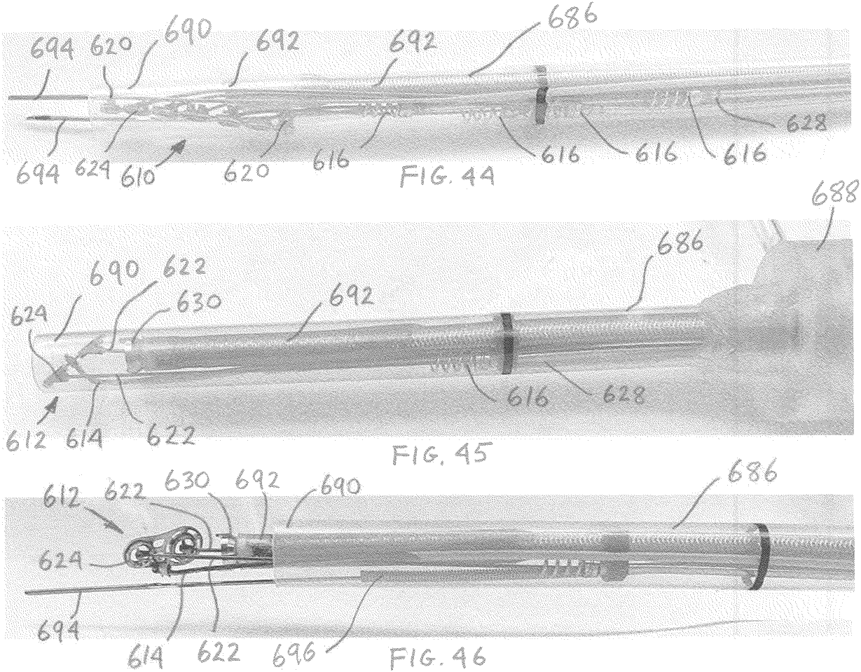

With the above-described arrangement, lead nut 666 may pivot with respect to spinner assembly 624, which in turn spins with respect to posterior implant 610 (shown in FIGS. 23, 24 and 37.) This allows an anchor lead 622 to generally lay flat against implant 610 when it is preloaded into a delivery catheter (as shown in FIG. 44), and to extend orthogonally or at another angle when the implant is being deployed. As best seen in FIGS. 33 and 40, one or more recesses 670 may be provided in the top of hoop 656 to allow connecting rod 664, lead nut 666 and anchor lead 622 (shown in FIGS. 24 and 37) to lay flatter against the implant.

Another advantage of spinner assemblies 624 is that they ensure that anchors 616 (shown in FIGS. 23 and 24) which thread through them are able to pull the implant all the way against the heart tissue without leaving any gaps between the tissue and the implant. The same spinner assemblies 624 may also be used with the two anterior implants 612 and work in much the same way as they do with posterior implant 610. As shown in FIGS. 23 and 24, a spinner assembly is provided for each anchor 616, so there are five spinner assemblies mounted on posterior implant 610 and two on each of the two anterior implants 612.

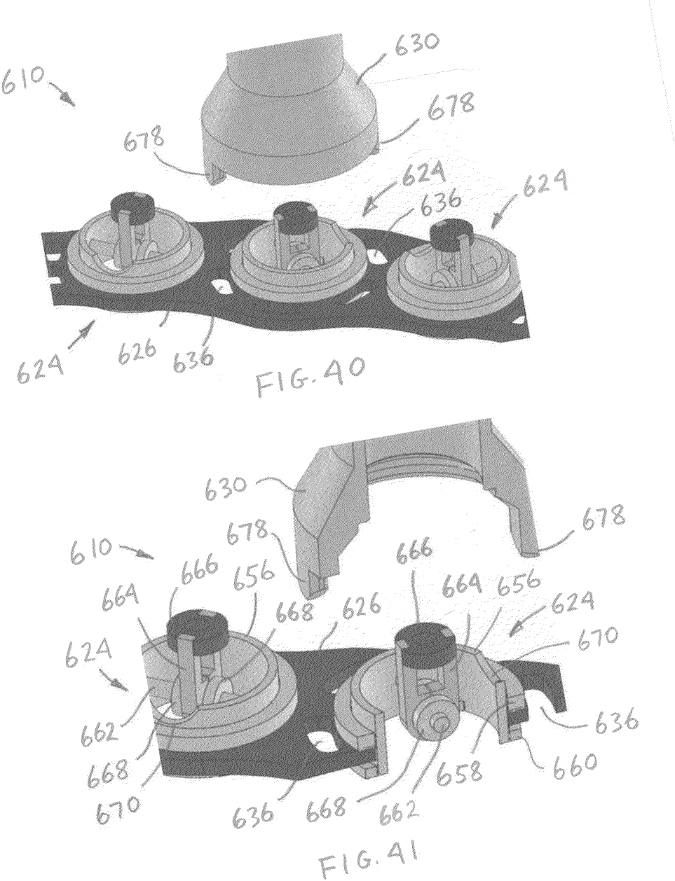

Referring to FIGS. 37-39, construction and operation of implantable anchors 616 will be described. In this exemplary embodiment, each anchor 616 is constructed from two components: a coil 672 and an anchor head 674. The distal end of anchor head 674 may be provided with a helical slot for receiving the proximal end of coil 672. In some embodiments, the proximal end of coil 672 is welded to head 674. In this exemplary embodiment, the center of anchor head 674 is hollow so that it fits over lead nut 666 and connecting rod 664 when anchor 616 is being implanted. The proximal end of anchor head 674 may be provided with a cylindrical, hook-shaped releasable engagement feature or clasp 676. An identical and or complementary mating feature or clasp 676 may be located on the distal end of driver head 628. When the implants are assembled and pre-loaded into delivery catheters, the two clasps 676 may be interlocked with each other and held together by an anchor lead 622. When interlocked, as shown in FIG. 38, clasps 676 transmit axial and rotation motion from driver head 628 to anchor 616 for driving the anchor through spinner assembly 624 and into the underlying heart tissue. After all the anchors 616 of an implant are installed, the distal end of each anchor lead 622 may be unscrewed from its associated lead nut 666 and withdrawn proximally through clasps 676 to allow each anchor driver to be disengaged from its anchor 616, as shown in FIG. 39. As shown in FIGS. 38 and 39, a series of slots or laser cuts 677 may be formed through the wall thickness of driver heads 628 to form flexures or living hinges. These flexures can relieve pressure and allow clasps 676 to more easily engage and disengage from one another when there is axial misalignment, or a lateral moment being applied to driver head 628. In other embodiments (not shown), a hollow stranded cable may be used instead of a rigid tube with or without flexures.

As also depicted in FIG. 37, torque head 630 may be provided with a flared distal end configured to fit over the center spinner assembly 624 when distally extending tabs 678 fit into slots 636 for steering implant 610. Torque head 630 may also be provided with a central bore large enough for an anchor 616 to be housed in when it is being installed.

Referring to FIGS. 40 and 41, additional views of torquer head 630 are shown. FIG. 40 shows the distal end of torquer head 630 as it approaches the central spinner assembly 624. Torquer head 630 may be engaged with implant 610 by pushing the proximal end of the torquer tube (not shown) in a distal direction while pulling the proximal end of central anchor lead 622 (shown in FIG. 23) in a proximal direction. The torquer tube may need to be rotated until tabs 678 engage with slots 636. FIG. 41 shows the distal end of torquer head 630 and central spinner assembly 624 with portions cut away to show further details of these components.

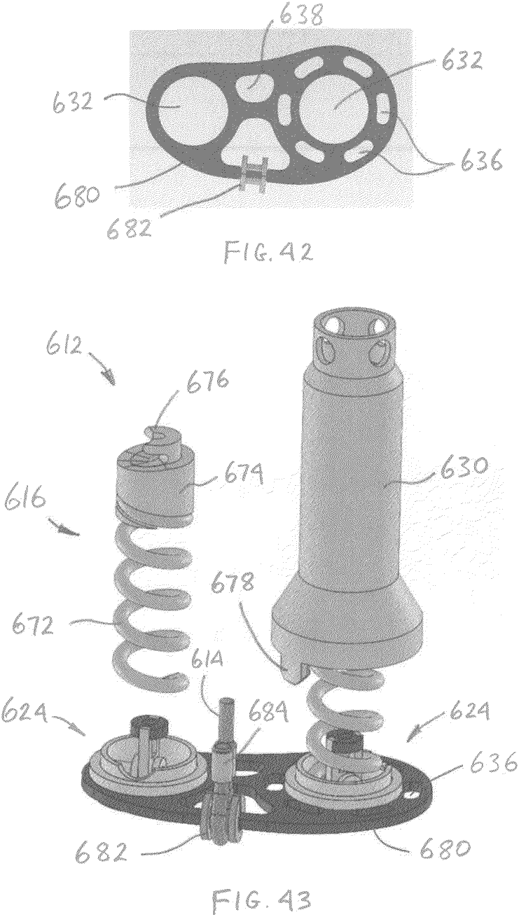

Referring to FIGS. 42 and 43, views of anterior implant 612 components are shown. FIG. 42 shows a bare anterior implant base plate 680 with only a tether thimble 682 attached. Since the two anterior implants 612 shown in FIGS. 23 and 24 are mirror images of one another, the same plate 680 and thimble 682 may be used to construct either one, depending on which direction spinner assemblies 624 are facing. When spinner assemblies 624 are mounted on the near side of plate 680 shown in FIG. 42 (such that the lead nuts 666 are pointed up, as shown in FIG. 43), a lateral anterior implant 612 is formed (shown on the left side of FIGS. 23 and 24.) When spinner assemblies 624 are mounted on the far side of plate 680 shown in FIG. 42 (such that the lead nuts 666 are pointed down, opposite of what is shown in FIG. 43), a medial anterior implant 612 is formed (shown on the right side of FIGS. 23 and 24.)

In this exemplary embodiment, identical components such the spinner assemblies 624, anchors 616, torque head 630, etc. are used for anterior implants 612 previously described for posterior implant 610. As shown in FIG. 42, the spacing of slots 636 may be the same as that used on the posterior implant plate 626 (shown in FIG. 25) for receiving the two opposing tabs 678 of torque head 630 (only one tab 678 seen in FIG. 43.) With slots 636 spaced every 60 degrees, torque head can be dithered no more than plus or minus 30 degrees or rotated no more than 60 degrees in one direction, before tabs 678 engage with a pair of mating slots 636.

As shown in FIG. 43, thimble 682 and sleeve 684 can be used to terminate the distal end of a tether 614 on a beam of an anterior implant 612 such that the tether can freely pivot relative to the implant. This can allow the implants 612 with their pre-attached tethers 614 to be more reliably loaded into and deployed from a delivery catheter. This pivoting also allows the tethers 614 to align directly towards the posterior implant 610 (as shown in FIGS. 23 and 24) rather than imparting rotational moments to the implanted anterior implants 612 and the underlying heart tissue.

In some embodiments, tethers or tensile members 614 have a composite structure. A continuous braided filament core may comprise an Ultra High Mechanical Polyethylene (UHMPE) fiber such as Dyneema® provided by Koninklijke DSM N.V. of the Netherlands, combined with a polyethylene terephthalate (PET) fiber. Dyneema® may be used for strength and durability and PET provides improved bonding with epoxy. In some embodiments, a 50%/50% combination of Dyneema® and PET is used. This continuous braided filament core may be inserted into or coated with a polyvinylidene fluoride (PVDF) jacketing to provide desirable handling characteristics, such as high column strength for threading the tether through a catheter and advancing the catheter without the tether collapsing. In some embodiments, at least one platinum wire is placed in the distal section of each tether 614 for radiopacity so that the tethers can be better seen under imaging. The filament core may be saturated with epoxy prior to being inserted into an outer jacket. This may be done to bind the composite together. In some embodiments, the outer jacket is run through a necking die to reduce its diameter and compress it into the filament. Tethers 614 may be color-coded so that the surgeons can distinguish a medial tether from a lateral tether. In some embodiments, markings are provided on the tethers 614 every 5 mm so that cinching may be observed. Applicants have discovered that the use of the above features provides the ability to cut the tethers in vivo, provides tethers with superior longitudinal stiffness for responsive cinching and superior durability for the life of the implants while supporting the full in vivo load of heart valve adjustment.

Referring to FIGS. 44-46, views of the exemplary implants are shown preloaded into delivery systems. In some embodiments, the implants are each loaded into their own implant loader 686. Implant loader 686 has a proximal hub 688 (shown in FIG. 45) configured to slide over the distal end of an inner steerable catheter (not shown.) The distal end 690 of implant loader 686 may be configured to slide within the proximal end of an outer steerable catheter (not shown.) During deployment of the implant, the implant loader 686 remains in place in the proximal end of the outer catheter while the implant and distal end of inner catheter slide distally through implant loader 686 and the outer catheter.

FIG. 44 shows a posterior implant 610 preloaded into an implant loader 686. As shown, implant 610 is close to parallel with the central axis of implant loader 686. Five anchor leads 622 are each attached to a spinner assembly 624 on the implant and lie generally flat against implant 610 when preloaded. Each anchor lead 622 extends proximally through an anchor 616 and attached anchor driver 628. Only four anchors 616 are visible, as the fifth anchor is located inside torque head 630.

A pair of tether pullers 694 may each be threaded through a spinning eyelet assembly 620 of posterior implant 610 and extend distally out of the implant loader 686 as shown. After the anterior implants are implanted, their tethers 614 may be attached to the protruding ends of the tether pullers 694, such as with a sleeve attached to each puller that can be crimped onto the tether. The tethers may then be pulled proximally with the tether pullers 694 until the proximal ends of the tethers emerge from the inner steerable catheter (not shown.)

FIG. 45 shows a medial anterior implant 612 preloaded into an implant loader 686. As shown, implant 612 forms an acute angle with the central axis of implant loader 686. Two anchor leads 622 are each attached to a spinner assembly 624 on the implant and extend proximally through an anchor 616 and attached anchor driver 628. Only one anchor 616 is visible, as the second anchor is located inside the torque head 630 torque tube 692.

FIG. 46 shows a lateral anterior implant 612 that has been preloaded into an implant loader 686 and is being pushed out of its distal end 690. In this exemplary embodiment, the preloading and deployment of the lateral anterior implant 612 is essentially the same as that of the medial anterior implant 612 shown in FIG. 45, but a tether puller 694 is provided for attaching to the proximal end of the tether from medial anterior implant 612 which is implanted first. In this exemplary embodiment, an axially extending spring lumen 696 is provided to guide tether puller 694. In this embodiment, spring lumen 696 resembles an automobile “curb feeler” in that it may be deflected from a straight orientation by a lateral force in operation but is biased to return to its original straight orientation. Spring lumen 696 serves to prevent tether puller 694 and later a tether itself from wrapping about the implant, another tether, lead or tube.

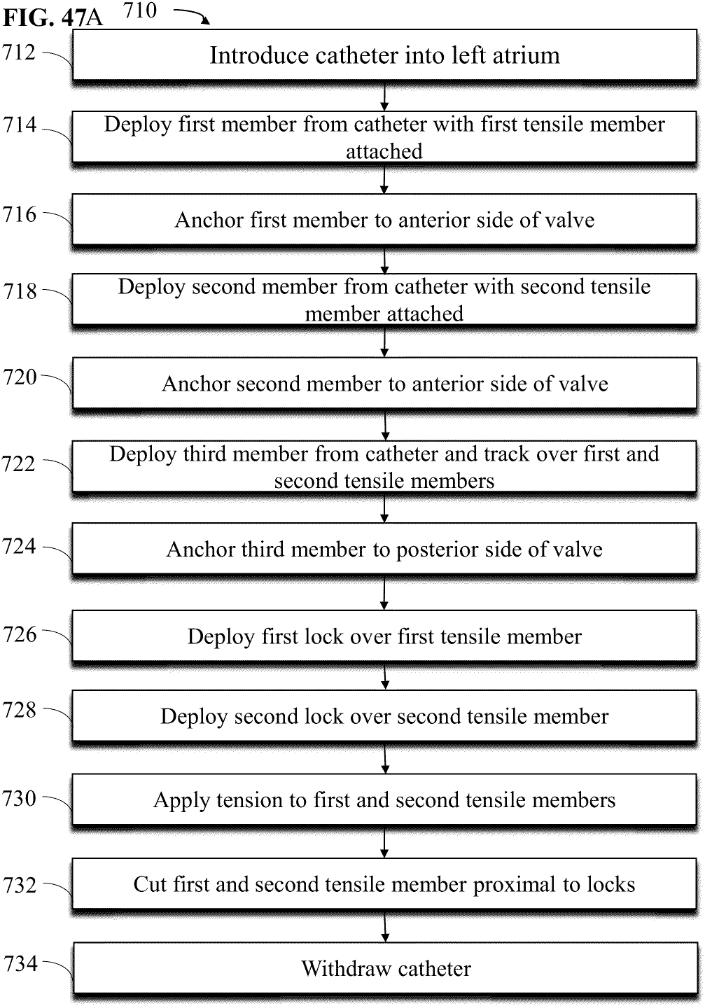

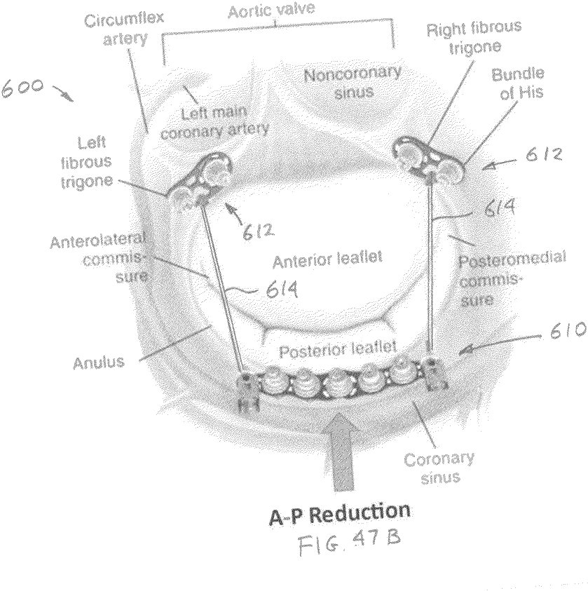

Referring to FIGS. 47A, 47B and 24, an exemplary method of performing an annuloplasty procedure according to aspects of the present disclosure is schematically shown. The steps of this exemplary method 710 are similar to those previously described in reference to the flowchart shown in FIG. 4 and the series of images shown in FIGS. 5-22. For ease of understanding, descriptions of details that are the same between the two methods will not be repeated below. In some implementations of the method, two anterior implants 612 and one posterior implant 610 are implanted. In other implementations, different types or numbers of devices may be used.

In some implementations of method 710, the first step 712 of the method is introducing the distal end of a steerable outer delivery catheter (not shown) into the left atrium of a subject. This may be performed using a transseptal approach, a left atrial approach or other methodology for gaining access to the left atrium. In some implementations, an inner dilator (not shown) is located in the distal end of the steerable outer delivery catheter for crossing the septum. A steerable inner delivery catheter (not shown) may be placed through the outer steerable delivery catheter for more precise delivery of the implants.

In step 714 of this exemplary embodiment, once the inner delivery catheter is introduced into the outer catheter, a first anterior implant 612 (see FIG. 24), sometimes referred to herein as a first member, and the distal end of the inner delivery catheter may be deployed into the left atrium from the distal end of the outer delivery catheter in. In this exemplary embodiment, a medial anterior device 612 is implanted first, followed by a lateral anterior device 612. In other embodiments, the order of implantation may be changed. Anchor leads 622 and the inner catheter may be used to push anterior implant 612 through the outer delivery catheter and deploy it from the distal end. After the first anterior implant 612 emerges from the distal end of the outer delivery catheter, anchor leads 622 may be manipulated from the proximal end of the inner delivery catheter to pivot anterior implant 612 into an orientation that is generally perpendicular to the inner delivery catheter. The first anterior implant 612 emerges from the outer delivery catheter with the distal end of its tether or first tensile member 614 pre-attached. The proximal end of the attached tether 614 extends through the delivery catheters and out the proximal ends. Torque head 630 may be slid distally over anchor lead 622 until distally extending tabs 678 fit into slots 636 (see FIG. 43) so that implant 612 may be steered into its desired implantation location and orientation. Alternatively, torque head 630 may remain stationary relative to the catheter and anchor lead 622 may be used to pull implant 612 proximally until it engages with torque head 630.

In step 716, anterior implant 612 (i.e., the first member) is anchored to the anterior side of the mitral valve. This may be accomplished by individually turning each of the two helical tissue anchors 616 with their attached driver heads 628. While torque head 630 holds anterior implant 612 against the mitral valve annulus tissue, one drive tube may be rotated to screw its anchor 616 through its spinner assembly 624 and into the underlying tissue. The torque head 630 may be used to then finely adjust/rotate implant 612 before the second anchor 616 is screwed into place with its drive tube and driver head 628. Proper placement of the first member may be confirmed through imaging. When the surgical staff is ready to remove the delivery instrumentation, leads 622 may be unscrewed from spinner assemblies 624, withdrawn past driver heads 628 and at least partially into the connected drive tubes. This allows the driver heads 628 to disengage from the anchors 616. Once driver heads 628 are disengaged, the attached drive tubes, anchor leads 622, torque head 630 and inner catheter can be proximally withdrawn through the outer delivery catheter.

In this exemplary embodiment, steps 718 and 720 are similar to steps 714 and 716, respectively. In step 718, a lateral anterior implant 612 (sometimes referred to herein as a second member) and the distal end of an inner delivery catheter may be deployed into the left atrium from the distal end of the outer delivery catheter in much the same way as previously described for the medial anterior implant 612 in step 714. In some embodiments, a separate, pre-loaded and pre-sterilized steerable inner catheter is provided for each of the anterior implants 612. In some embodiments, the tether 614 from the previously implanted first member 612 remains in the outer catheter after the inner catheter for the first member has been removed. To avoid entanglement, this tether 614 may be threaded through the second inner catheter before the second inner catheter is introduced into the outer steerable catheter. In this exemplary embodiment, the second member 612 is deployed with its own tether or tensile member 614 attached, such that the proximal ends of both the first and second tethers 614 extend through the second inner catheter and out of its proximal end. In step 720, the lateral anterior implant 612 (i.e., the second member) is anchored to the anterior side of the valve in much the same way as previously described for the medial anterior implant 612 (i.e., the first member.)

In step 722, posterior implant 610 (sometimes referred to herein as a third member) is deployed into the heart. As with the first and second members, the third member may be provided to the surgeons preloaded into its own steerable inner catheter. In some embodiments, before the third inner catheter is introduced into the outer catheter, the tethers or tensile members 614 extending from the first and second members are threaded through eyelet assemblies 620 on posterior implant 610 and through the third inner catheter. Anchor leads 622 and the inner catheter may be used to push anterior implant 612 through the outer delivery catheter and deploy it from the distal end. After the posterior implant 610 emerges from the distal end of the outer delivery catheter, anchor leads 622 may be manipulated from the proximal end of the inner delivery catheter to pivot posterior implant 610 into an orientation that is generally perpendicular to the inner delivery catheter. By keeping some tension on the proximal ends of the tethers 614 connected to the implanted first and second members, the posterior implant 610 or third member emerges from the outer delivery catheter and tracks over the first and second tensile members 614. One advantage to this arrangement is that the first and second tensile members 614 help guide the third member 610 into a proper orientation. Having the tensile members 614 pre-connected to the three implants also saves time during the surgical procedure and ensures that the tensile members are properly and consistently connected to the implants. Torque head 630 may be slid distally over the center anchor lead 622 until distally extending tabs 678 fit into slots 636 (see FIG. 40) so that implant 610 may be further steered into its desired implantation location and orientation. Alternatively, torque head 630 may remain stationary relative to the catheter and anchor lead 622 may be used to pull implant 610 proximally until it engages with torque head 630.

In step 724, posterior implant 610 (i.e., the third member) is anchored to the posterior side of the mitral valve. This may be accomplished by individually turning each of the five helical tissue anchors 616 with their attached driver heads 628. While torque head 630 holds posterior implant 610 against the mitral valve annulus tissue, one drive tube may be rotated to screw its anchor 616 through its spinner assembly 624 and into the underlying tissue. The torque head 630 may be used to then finely adjust/rotate implant 610 before the next anchors 616 are screwed into place with their drive tubes and driver heads 628. Proper placement of the third member may be confirmed through imaging. When the surgical staff is ready to remove the delivery instrumentation, leads 622 may be unscrewed from spinner assemblies 624, withdrawn past driver heads 628 and at least partially into the connected drive tubes. This allows the driver heads 628 to disengage from the anchors 616. Once driver heads 628 are disengaged, the attached drive tubes, anchor leads 622, torque head 630 and inner catheter can be proximally withdrawn through the outer delivery catheter.

In other embodiments, the order of deployment and attachment of the multiple implants can be changed. In these other embodiments, the first implant or implants are deployed with tether(s) attached, and at least one subsequently deployed implant tracks over the tether(s) when it is being deployed and attached to heart tissue. For example, a posterior implant may be implanted first with two tethers pre-attached at opposite ends of the implant. A medial anterior implant may then be deployed, tracking over one of the tethers of the posterior implant. After the medial anterior implant is secured to underlying heart tissue, a lateral anterior implant may be deployed, tracking over the other tether of the posterior implant. In another embodiment, a single anterior implant with two tethers is implanted first, and a single posterior implant may then be deployed, tracking over the two tethers of the anterior implant. Other embodiments having different orders of implant deployment may also utilize the principles of the present disclosure.

In exemplary method 710, once all three implants have been deployed and anchored, additional tension may be applied to the interconnecting tethers 614 to draw the anterior and posterior sides of the mitral valve into closer approximation. This may be accomplished in steps 726, 728 and 730 of method 710. In step 726, a first lock 618 is deployed over the first tensile member 614 which is connected to the medial anterior implant 612. In step 728, a second lock 618 is deployed over the second tensile member 614 which is connected to the lateral anterior implant 612. The locks 618 may be pushed over the tensile members 614 from their proximal ends by sleeve-like tools (not shown) that urge the locks 618 distally until they abut against eyelet assemblies 620, as shown in FIGS. 23 and 24. In step 730, tension is then applied to the first and second tensile members 614 by pulling proximally on the tensile members while pushing distally against locks 618 with the sleeve-like tools.

In some implementations, tension in tensile members 614 may be increased simultaneously. In some implementations, tension may be increased incrementally in members 614, alternating between the two until the desired tensions and or valve approximation is reached. In some implementations, the final tension and or tissue approximation of each tensile member 614 is approximately the same. In some implementations, the final tension and or tissue approximation of each tensile member 614 is different. Because medial and lateral cinching can be performed independently, the placement of each implant is more forgiving. In some implementations, real time echocardiography of the mitral valve is used to monitor the reduction in mitral regurgitation as tensile members 614 are tightened. In some embodiments, one or both locks 618 may be temporarily released if it is desired to reduce the tension in the tensile members 614.

After the desired tensions and or tissue approximations are obtained, the excess length of tensile members 614 extending proximally from locks 618 may be cut off. In step 732, a cutter assembly may be slid distally along each tensile member 614 until it reaches the lock 618. It may then be activated to cut the tensile member and then withdrawn with the cut off portion of the tensile member. In step 734, the outer delivery catheter may then be withdrawn from the left atrium,

In some embodiments, the systems and methods disclosed herein or portions thereof may be utilized in a similar manner on either atrioventricular valve.

Advantages provided by the systems and methods disclosed herein can include the following. A more direct reduction in the anterior-posterior (A-P) direction can be achieved. Because the A-P direction is the most clinically relevant dimension to be reduced, it is advantageous to directly affect this dimension as opposed to simultaneously changing other dimensions of the annulus. This can be accomplished with a reduced cinching force because the action is directly in the A-P direction, rather than larger forces that are generally needed with circumferential remodeling. Lower cinching force typically translates to fewer anchors required. The systems and methods also allow for a high level of customization to suit a particular anatomy. This relates to there being distinct components that are placed separately, and the ability to adjust the medial and lateral sides separately. The separate components are each easier to implant compared with one superstructure. Each of the components can be retrieved prior to anchor detachment. The systems and methods allow for in vivo adjustability, allow for reduced accuracy needed to place the components and simplify the implantation procedure. A smaller number of implant sizes and configurations can also be accommodated. In addition to tensioning the device during the de novo procedure, additional tensioning devices can be added at a later time or date, and or the existing devices can be re-tensioned to further reduce the A-P dimension.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about”, “approximately” or “generally” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable annuloplasty system comprising:
a first member configured to be anchored to an anterior side of a mitral valve;
a first flexible tensile member attached to the first member;
a second member configured to be anchored to an anterior side of a mitral valve;
a second flexible tensile member attached to the second member;
a third member configured to be anchored to a posterior side of a mitral valve, the third member having a first attachment component configured to slidably attach to the first flexible tensile member, the third member having a second attachment component configured to slidably attach to the second flexible tensile member, the first and the second attachment components enabling the third member to slidably track over the first and the second flexible tensile members when the third member is being deployed from a catheter into a left atrium;
a first lock movable between a sliding state and a locked state, the first lock being configured to slide over the first flexible tensile member and abut against the first attachment component when in the sliding state and hold tension in the first flexible tensile member when in the locked state; and
a second lock movable between a sliding state and a locked state, the second lock being configured to slide over the second flexible tensile member and abut against the second attachment component when in the sliding state and hold tension in the second flexible tensile member when in the locked state.

2. The implantable annuloplasty system of claim 1, wherein the first member is configured to be deployed from a catheter into the left atrium with the first flexible tensile member pre-attached, and wherein the second member is configured to be deployed from a catheter into the left atrium with the second flexible tensile member pre-attached.

3. The implantable annuloplasty system of claim 1, wherein the first and the second attachment components each comprise an eyelet.

4. The implantable annuloplasty system of claim 3, wherein the eyelets are configured to rotate relative to the third member.

5. The implantable annuloplasty system of claim 1, wherein the third member has an elongated shape and the first and the second attachment components are located on opposite ends of the third member.

6. The implantable annuloplasty system of claim 1, wherein the first, the second and the third members each comprise at least one anchor attachment point configured to releasably attach to an anchor lead, wherein the anchor attachment points are configured to pivot and spin relative to the first, the second and the third members, thereby allowing the anchor leads to lie flat against the members when the members are being deployed from a catheter.

7. The implantable annuloplasty system of claim 6, wherein the attachment points each comprise a lead nut configured to threadably engage with one of the anchor leads, the lead nut being mounted on a connecting rod that is pivotably attached to crossbar, and the crossbar spanning a central hoop that is rotatably mounted on the first, the second or the third member.

8. The implantable annuloplasty system of claim 6, wherein the first, the second and the third members each comprise at least one anchor configured to anchor the member to underlying heart tissue, wherein each of the anchors is configured to release from an attached driver head when one of the anchor leads is released from its anchor attachment point and proximally withdrawn through the anchor and the driver head.

9. The implantable annuloplasty system of claim 8, wherein each of the anchors comprises a cylindrical, hook-shaped clasp configured to releasably engage with a mating clasp on one of the driver heads.

10. The implantable annuloplasty system of claim 1, wherein the first and the second flexible tensile members each have a braided filament core and wherein the braided filament core is covered or coated with a polymer jacket.

11. The implantable annuloplasty system of claim 1, wherein the first and the second flexible tensile members each have a continuous braided filament core comprising an ultra high mechanical polyethylene (UHMPE) fiber combined with a polyethylene terephthalate (PET) fiber, and wherein the continuous braided filament core is covered with a polyvinylidene fluoride (PVDF) jacketing.

12. The implantable annuloplasty system of claim 1, wherein the first flexible tensile member comprises a color that is different from that of the second flexible tensile member such that the first flexible tensile member can be distinguished from the second flexible tensile member by color during a surgical procedure.

13. An implantable annuloplasty system comprising:
a first member configured to be anchored to an anterior side of a mitral valve;
a first flexible tensile member attached to the first member, wherein the first member is configured to be deployed from a catheter into the left atrium with the first flexible tensile member pre-attached;
a second member configured to be anchored to an anterior side of a mitral valve;
a second flexible tensile member attached to the second member, wherein the second member is configured to be deployed from a catheter into the left atrium with the second flexible tensile member pre-attached;
an elongated third member configured to be anchored to a posterior side of a mitral valve, the third member having a first eyelet configured to slidably receive the first flexible tensile member, the third member having a second eyelet configured to slidably receive the second flexible tensile member, the first and the second eyelets being located on opposite ends of the third member and each being configured to rotate relative to the third member, the first and the second eyelets enabling the third member to slidably track over the first and the second flexible tensile members when the third member is being deployed from a catheter into a left atrium;
a first lock movable between a sliding state and a locked state, the first lock being configured to slide over the first flexible tensile member and abut against the first eyelet when in the sliding state and hold tension in the first flexible tensile member when in the locked state; and
a second lock movable between a sliding state and a locked state, the second lock being configured to slide over the second flexible tensile member and abut against the second eyelet when in the sliding state and hold tension in the second flexible tensile member when in the locked state,
wherein the first, the second and the third members each comprise at least one anchor attachment point configured to releasably attach to an anchor lead, wherein the anchor attachment points are configured to pivot and spin relative to the first, the second and the third members, thereby allowing the anchor leads to lie flat against the members when the members are being deployed from a catheter,
wherein the attachment points each comprise a lead nut configured to threadably engage with one of the anchor leads, the lead nut being mounted on a connecting rod that is pivotably attached to crossbar, and the crossbar spanning a central hoop that is rotatably mounted on the first, the second or the third member,
wherein the first, the second and the third members each comprise at least one anchor configured to anchor the member to underlying heart tissue, wherein each of the anchors is configured to release from an attached driver head when one of the anchor leads is released from its anchor attachment point and proximally withdrawn through the anchor and the driver head,
wherein each of the anchors comprises a cylindrical, hook-shaped clasp configured to releasably engage with a mating clasp on one of the driver heads,
wherein the first and the second flexible tensile members each have a braided filament core and wherein the braided filament core is covered or coated with a polymer jacket, and
wherein the first flexible tensile member comprises a color that is different from that of the second flexible tensile member such that the first flexible tensile member can be distinguished from the second flexible tensile member by color during a surgical procedure.

14. A method for performing an annuloplasty procedure, the method comprising:
introducing a catheter into a left atrium of a heart;
deploying a first member from the catheter;
anchoring the first member to an anterior side of a mitral valve in the left atrium, the first member having a first flexible tensile member attached;
deploying a second member from the catheter;
anchoring the second member to the anterior side of the mitral valve in the left atrium, the second member having a second flexible tensile member attached;
deploying a third member from the catheter, wherein the third member slidably tracks over the first and the second flexible tensile members;
anchoring the third member to a posterior side of the mitral valve in the left atrium; and
applying tension to the first and the second tensile members to draw the first member and the second member toward the third member, thereby bringing the posterior side and the anterior side of the mitral valve annulus into closer approximation.

15. The method of claim 14, wherein the first member is deployed from the catheter with the first flexible tensile member pre-attached, and wherein the second member is deployed from the catheter with the second flexible tensile member pre-attached.

16. The method of claim 14, wherein the step of applying tension to the first and the second tensile members comprises deploying a first lock over the first tensile member and deploying a second lock over the second tensile member.

17. The method of claim 14, further comprising the step of cutting the first tensile member proximal to the first lock and cutting the second tensile member proximal to the second lock.

18. The method of claim 14, wherein the step of applying tension to the first and the second tensile members comprises applying tension independently to the two separate tensile members.

19. The method of claim 14, wherein the first member is anchored toward a medial side of the mitral valve and the second member is anchored toward a lateral side of the mitral valve.

20. The method of claim 19, wherein the first member has at least one anchor within proximity of the medial trigon and the second member has at least one anchor within proximity of the lateral trigon.

21. The method of claim 19, wherein at least one of the steps of anchoring the first member and the second member comprises attaching at least two separate anchors by screwing the separate anchors into the mitral valve annulus.

22. The method of claim 19, wherein a dimensional reduction of the mitral valve annulus in an anterior-posterior direction can be different on the lateral side and the medial side.

23. The method of claim 14, wherein the third member has an elongated shape, and wherein the method further comprises rotating the elongated third member into a desired position before anchoring it to the posterior side of the mitral valve annulus.

24. The method of claim 14, wherein the first member, the second member and the third member are each deployed from the catheter with at least one anchor lead attached.

25. An implantable heart therapy system comprising:
a first member configured to be anchored to a first side of a heart valve;
a second member configured to be anchored to an opposite side of the heart valve; and
a flexible tensile member spanning between the first and the second members, wherein the flexible tensile member has a continuous braided filament core comprising an ultra high mechanical polyethylene (UHMPE) fiber combined with a polyethylene terephthalate (PET) fiber, and wherein the continuous braided filament core is covered with a polyvinylidene fluoride (PVDF) jacketing.
